(19) United States
(12) Patent Application Publication
(10) Pub. No.: US 2023/0122601 A1
(43) Pub. Date: Apr. 20, 2023

(54) Self-Calibration for Pump Operation of Medical Injector

(71) Applicant: Becton, Dickinson and Company, Franklin Lakes, NJ (US)

(72) Inventors: Simon O'Reilly, Dublin (IE); Kun Li, Boyds, MD (US); Scott Corey, Hydes, MD (US)

(21) Appl. No.: 17/967,323

(22) Filed: Oct. 17, 2022

Related U.S. Application Data
(60) Provisional application No. 63/256,851, filed on Oct. 18, 2021.

(51) Int. Cl. A61M 5/168; A61M 5/145

(52) U.S. Cl. CPC A61M 5/16854; A61M 5/145; A61M 2205/3331

(57) ABSTRACT

A method of pressure management of a drug delivery device including a pump, a fluid line, and a power source, includes: actuating the pump a predetermined number of cycles to move fluid through the fluid line, wherein the predetermined number of cycles is greater than one; determining an estimated pressure value within the fluid line for each cycle of the predetermined number of cycles; and determining a baseline pressure level comprising an average of the estimated pressure values.

### CROSS-REFERENCE TO RELATED APPLICATION

The present application claims priority to U.S. Provisional Application No. 63/256,851, entitled “Self-Calibration for Pump Operation of Medical Injector”, filed Oct. 18, 2021, the entire disclosure of which is hereby incorporated by reference in its entirety.

### BACKGROUND OF THE INVENTION

### Field of the Invention

The present disclosure relates to a method of self-calibrating a pump operation of a medical injector.

### Description of Related Art

Wearable medical devices, such as automatic injectors, have the benefit of providing therapy to the patient at a location remote from a clinical facility and/or while being worn discretely under the patient's clothing. The wearable medical device can be applied to the patient's skin and configured to automatically deliver a dose of a pharmaceutical composition within a predetermined time period after applying the wearable medical device to the patient's skin, such as after a 27 hour delay. After the device delivers the pharmaceutical composition to the patient, the patient may subsequently remove and dispose of the device.

In certain circumstances, due to the medium in which the liquid is being injected, the flow of fluid leaving the device may be impaired, which can lead to increased pressure in the fluid line of the device. When the pressure rises above a certain threshold, the integrity of the fluid path may be compromised causing a leak within the device and a failure to deliver the full dose of medicament. A fluid leak within the device may also cause damage to the device and subsequent system failures as well as potential contamination concerns due to contact between the fluid and the device.

Human subcutaneous tissue is composed of various cell types, extracellular matrix (ECM) constituents, microstructures, and macroscopic arrangement of cells and ECM. Those elements contribute to the mechanical properties of the tissue. The tissue may also include lymphatic system and blood vessels, and has intrinsic fluid absorption and retention properties. These characteristics vary among individuals, location within the body, and over time may cause variable degrees of resistance to the infusion of fluids at the site of injection. When the resistance of the tissue is too high or the absorption rate is too low for a given delivery flow rate from the device, the pressure may build up and reach valves above the threshold where the fluid line and other components may be compromised.

### SUMMARY OF THE INVENTION

In one aspect or embodiment, a method of pressure management of a drug delivery device including a pump, a fluid line, and a power source, includes: actuating the pump a predetermined number of cycles to move fluid through the fluid line, wherein the predetermined number of cycles is greater than one; determining an estimated pressure value within the fluid line for each cycle of the predetermined number of cycles; and determining a baseline pressure level comprising an average of the estimated pressure values.

The drug delivery device may include a cannula, where the method is performed prior to insertion of the cannula into a patient. Determining the estimated pressure value may include measuring a current of a motor during actuation of the pump. Determining the baseline pressure level may include excluding each estimated pressure value where the motor current differs from an average of the measured motor currents of the estimated pressure values by more than 1 mA. The drug delivery device may include a reservoir, where the method is performed prior to filling the reservoir with a medicament. The method may further include: delivering fluid through the fluid line via the pump; determining a delivery pressure value within the fluid line; and determining whether the delivery pressure value exceeds the baseline pressure level. Determining a delivery pressure value within the fluid line may include measuring a current of a motor during actuation of the pump. The method may be performed using a microcontroller including at least one processor.

In a further aspect or embodiment, a drug delivery device includes: a power source; a reservoir configured to receive a fluid; a fluid line in fluid communication with the reservoir; a pump configured to deliver the fluid from the reservoir to the fluid line; and a microcontroller comprising at least one processor programmed or configured to cause the device to: actuate the pump a predetermined number of cycles to move fluid through the fluid line, where the predetermined number of cycles is greater than one; determine an estimated pressure value within the fluid line for each cycle of the predetermined number of cycles; and determine a baseline pressure level comprising an average of the estimated pressure values.

The at least one processor may be further programmed or configured to: deliver fluid through the fluid line via the pump; determine a delivery pressure value within the fluid line; and determine whether the delivery pressure value exceeds the baseline pressure level.

In a further aspect or embodiment, a computer program product for a method of pressure management for a drug delivery device comprising a microcontroller, a reservoir, a pump, a fluid line, and a power source, includes at least one non-transitory computer-readable medium including program instructions that, when executed by the microcontroller, cause the drug delivery device to: actuate the pump a predetermined number of cycles to move fluid through the fluid line, wherein the predetermined number of cycles is greater than one; determine an estimated pressure value within the fluid line for each cycle of the predetermined number of cycles; and determine a baseline pressure level comprising an average of the estimated pressure values.

The at least one non-transitory computer-readable medium may further include program instructions that, when executed by the microcontroller, cause the microcontroller to: deliver fluid through the fluid line via the pump; determine a delivery pressure value within the fluid line; and determine whether the delivery pressure value exceeds the baseline pressure level.

### BRIEF DESCRIPTION OF THE DRAWINGS

The above-mentioned and other features and advantages of this disclosure, and the manner of attaining them, will become more apparent and the disclosure itself will be better understood by reference to the following descriptions of embodiments of the disclosure taken in conjunction with the accompanying drawings, wherein:

FIG. 1 is a perspective view of a drug delivery device according to one aspect or embodiment of the present application.

FIG. 2 is a perspective view of the drug delivery device of FIG. 1, with a top cover removed.

FIG. 3 is a schematic of the drug delivery device of FIG. 1.

FIG. 4 is a schematic of a method of pressure management of a drug delivery device according to one aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. By “about” is meant a range of plus or minus ten percent of the stated value. As used in the specification and the claims, the singular form of “a”, “an”, and “the” include plural referents unless the context clearly dictates otherwise. The terms “first”, “second”, and the like are not intended to refer to any particular order or chronology, but instead refer to different conditions, properties, or elements. By “at least” is meant “greater than or equal to”.

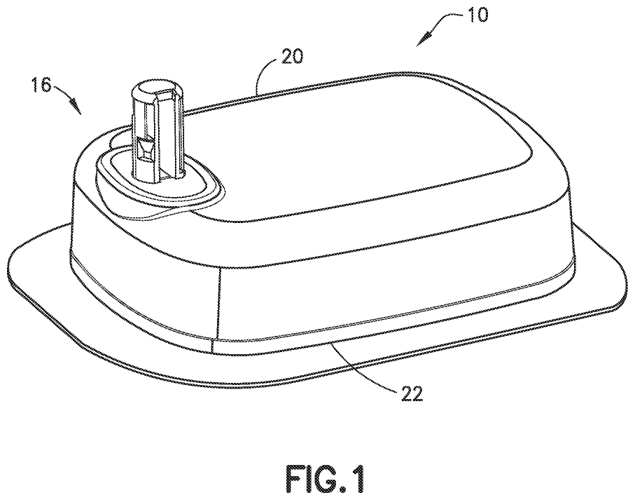

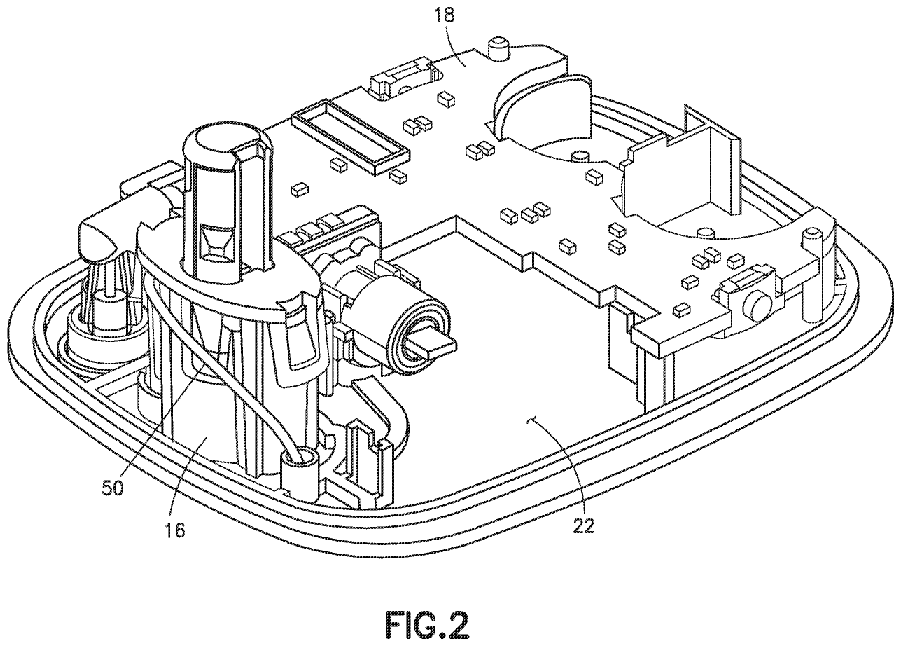

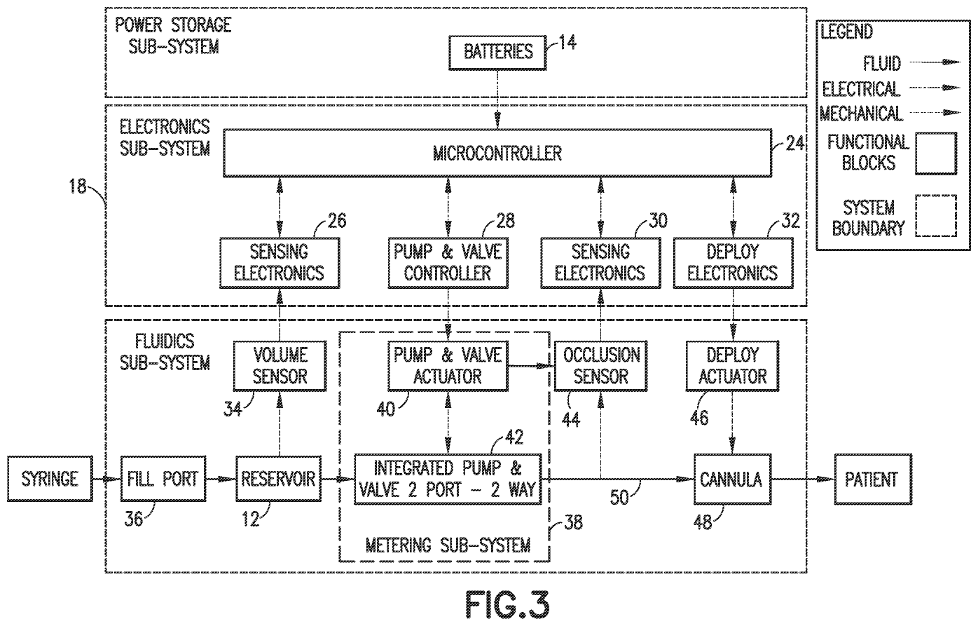

Referring to FIGS. 1-3, a drug delivery device 10 includes a reservoir 12, a power source 14, an insertion mechanism 16, control electronics 18, a cover 20, and a base 22. In one aspect or embodiment, the drug delivery device 10 is a wearable automatic injector, such as an insulin or bone marrow stimulant delivery device. The drug delivery device 10 may be mounted onto the skin of a patient and triggered to inject a pharmaceutical composition from the reservoir 12 into the patient. The drug delivery device 10 may be pre-filled with the pharmaceutical composition, or it may be filled with the pharmaceutical composition by the patient or medical professional prior to use.

The drug delivery device 10 is configured to deliver a dose of a pharmaceutical composition, e.g., any desired medicament, into the patient's body by a subcutaneous injection at a slow, controlled injection rate. Exemplary time durations for the delivery achieved by the drug delivery device 10 may range from about 5 minutes to about 60 minutes, but are not limited to this exemplary range. Exemplary volumes of the pharmaceutical composition delivered by the drug delivery device 10 may range from about 0.1 milliliters to about 10 milliliters, but are not limited to this exemplary range. The volume of the pharmaceutical composition delivered to the patient may be adjusted.

Referring again to FIGS. 1-4, in one aspect or embodiment, the power source 14 is a DC power source including one or more batteries. The control electronics 18 include a microcontroller 24, sensing electronics 26, a pump and valve controller 28, sensing electronics 30, and deploy electronics 32, which control the actuation of the drug delivery device 10. The drug delivery device 10 includes a fluidics sub-system that includes the reservoir 12, volume sensor 34 for the reservoir 12, a reservoir fill port 36, and a metering system 38 including a pump and valve actuator 40 and a pump and valve mechanism 42. The fluidic sub-system may further include an occlusion sensor 44, a deploy actuator 46, a cannula 48 for insertion into a patient's skin, and a fluid line 50 in fluid communication with the reservoir 12 and the cannula 48. In an alternative aspect or embodiment, the occlusion sensor is a system for measuring the motor current. In one aspect or embodiment, the insertion mechanism 16 is configured to move the cannula 48 from a retracted position positioned entirely within the device 10 to an extended position where the cannula 48 extends outside of the device 10. The drug delivery device 10 may operate in the same manner as discussed in U.S. Pat. No. 10,449,292 to Pizzochero et al.

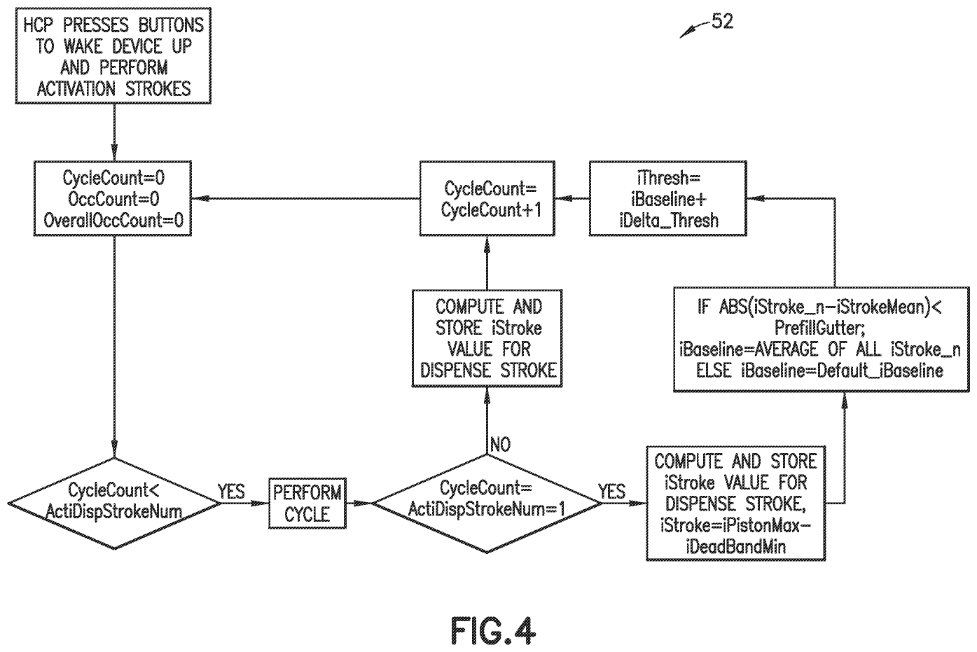

Referring to FIG. 4, in one aspect or embodiment, a method 52 of pressure management of the drug delivery device 10 includes: actuating the pump 42 a predetermined number of cycles to move fluid through the fluid line 50, where the predetermined number of cycles is greater than one; determining an estimated pressure value within the fluid line 50 for each cycle of the predetermined number of cycles; and determining a baseline pressure level comprising an average of the estimated pressure values. In one aspect or embodiment, the predetermined number of cycles is three or more pump cycles.

In one aspect or embodiment, the method 52 is performed prior to insertion of the cannula 48 into a patient and/or prior to filling the reservoir 12 with a fluid or medicament. The estimated pressure value is determined by measuring a current of a motor of the pump 40 during actuation of the pump 42.

In one aspect or embodiment, the baseline pressure level is determined by excluding each estimated pressure value where the motor current differs from an average of the measured motor currents of the estimated pressure values by more than 1 mA. Excluding such estimated pressure values filters out any estimated pressure values that may have been affected by undetermined noise factors.

In one aspect or embodiment, the method 52 further includes delivering fluid through the fluid line 50 via the pump 42; determining a delivery pressure value within the fluid line 50; and determining whether the delivery pressure value exceeds the baseline pressure level. In one aspect or embodiment, the delivery pressure value is determined by measuring a current of a motor of the pump 40 during actuation of the pump 42. The method 52 may be performed using the microcontroller 24, including the use of at least one processor.

As noted above, the method 52 may be performed prior to the device 10 being filled within a medicament and prior to being placed on a patient, but after manufacture and inspection of the device 10. Accordingly, at this stage of the device 10, there is a high probability the device 10 is not blocked or occluded while the method 52 is performed. In one aspect or embodiment, the baseline pressure level represents a minimum level to operate the pump 42, i.e., when the pump 42 is not blocked or occluded, such that any increase above the baseline pressure level can be attributed to the pump 42 having to operate with at least a partially occluded fluid path. In one aspect or embodiment, the method 52 is performed during priming of the device 10.

The algorithm logic of the method 52 is shown in FIG. 4. The method 52 may include one or more iterative processes from the beginning of the method 52 to delivery of a dose of fluid with the device 10.

In one aspect or embodiment, the pressure within the fluid line 50 is determined by measuring a current of the drug delivery device 10 during actuation of the pump 42. The measuring of the current of the drug delivery device 10 may include subtracting a reference or baseline current value from a peak current value during an actuation cycle of the pump 42 to determine a stroke current value, although other suitable current detection arrangements may be utilized. The stroke current value is utilized to estimate the downstream pressure of the fluid line 50 for the particular actuation cycle of the pump 42. For example, the stroke current value can be corresponded to various downstream pressure levels through testing or benchmarking such that the stroke current value can be used to estimate the pressure level of the fluid line 50.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

The invention claimed is:

1. A method of pressure management of a drug delivery device comprising a pump, a fluid line, and a power source, the method comprising:
a) actuating the pump a predetermined number of cycles to move fluid through the fluid line, wherein the predetermined number of cycles is greater than one;
b) determining an estimated pressure value within the fluid line for each cycle of the predetermined number of cycles; and
c) determining a baseline pressure level comprising an average of the estimated pressure values.

2. The method of claim 1, wherein the drug delivery device comprises a cannula, and wherein the method is performed prior to insertion of the cannula into a patient.

3. The method of claim 1, wherein determining the estimated pressure value comprises measuring a current of a motor during actuation of the pump.

4. The method of claim 3, wherein determining the baseline pressure level comprises excluding each estimated pressure value where the motor current differs from an average of the measured motor currents of the estimated pressure values by more than 1 mA.

5. The method of claim 1, wherein the drug delivery device comprises a reservoir, and wherein the method is performed prior to filling the reservoir with a medicament.

6. The method of claim 5, further comprising:
d) delivering fluid through the fluid line via the pump;
e) determining a delivery pressure value within the fluid line; and
f) determining whether the delivery pressure value exceeds the baseline pressure level.

7. The method of claim 6, wherein determining a delivery pressure value within the fluid line comprises measuring a current of a motor during actuation of the pump.

8. The method of claim 1, wherein a) through c) are performed using a microcontroller comprising at least one processor.

9. A drug delivery device comprising:
a power source;
a reservoir configured to receive a fluid;
a fluid line in fluid communication with the reservoir;
a pump configured to deliver the fluid from the reservoir to the fluid line; and
a microcontroller comprising at least one processor programmed or configured to cause the device to:
actuate the pump a predetermined number of cycles to move fluid through the fluid line, wherein the predetermined number of cycles is greater than one;
determine an estimated pressure value within the fluid line for each cycle of the predetermined number of cycles; and
determine a baseline pressure level comprising an average of the estimated pressure values.

10. The device of claim 9, wherein the at least one processor is further programmed or configured to:
deliver fluid through the fluid line via the pump;
determine a delivery pressure value within the fluid line; and
determine whether the delivery pressure value exceeds the baseline pressure level.

11. A computer program product for a method of pressure management for a drug delivery device comprising a microcontroller, a reservoir, a pump, a fluid line, and a power source, the computer program product comprising at least one non-transitory computer-readable medium including program instructions that, when executed by the microcontroller, cause the drug delivery device to:
actuate the pump a predetermined number of cycles to move fluid through the fluid line, wherein the predetermined number of cycles is greater than one;
determine an estimated pressure value within the fluid line for each cycle of the predetermined number of cycles; and
determine a baseline pressure level comprising an average of the estimated pressure values.

12. The computer program product of claim 11, wherein the at least one non-transitory computer-readable medium further includes program instructions that, when executed by the microcontroller, cause the microcontroller to:
deliver fluid through the fluid line via the pump;
determine a delivery pressure value within the fluid line; and
determine whether the delivery pressure value exceeds the baseline pressure level.
